(19) United States
(12) Patent Application Publication
(10) Pub. No.: US 2020/0078347 A1
(43) Pub. Date: Mar. 12, 2020

(54) THERAPEUTIC AGENT FOR ONYCHOMYCOSIS

(71) Applicant: KANEKA CORPORATION, Osaka-shi (JP)

(72) Inventors: Masaya MIZUTANI, Hyogo (JP); Tatsuyoshi TANAKA, Hyogo (JP); Hiroyuki OGINO, Hyogo (JP); Mitsuji AKAZAWA, Hyogo (JP)

(73) Assignee: KANEKA CORPORATION, Osaka-shi (JP)

(21) Appl. No.: 16/469,863

(22) PCT Filed: Dec. 15, 2017
(86) PCT No.: PCT/JP2017/045062

§ 371 Date: Jun. 14, 2019

(30) Foreign Application Priority Data
Dec. 16, 2016 (JP) 2016-244478

(51) Int. Cl. A61K 31/454; A61K 47/38; A61K 47/14; A61K 47/10; A61P 31/10

(52) U.S. Cl. CPC A61K 31/454; A61K 47/38; A61P 31/10; A61K 47/10; A61K 47/14

(57) ABSTRACT

It is an object of the present invention to provide an efinaconazole-containing film-forming preparation, which has no sticky surface after solidification thereof, has sufficient cohesiveness after solidification thereof, and is easily peeled without washing. The present invention provides a therapeutic preparation for onychomycosis, comprising efinaconazole or a salt thereof, 1% by weight or more and 30% by weight or less of ethyl cellulose, 20% by weight or more of a volatile solvent (wherein the volatile solvent is water or a liquid component having a boiling point lower than that of water), and 0.01% by weight or more and less than 30% by weight of a surfactant.

### TECHNICAL FIELD

The present invention relates to a preparation that can be used for the treatment of onychomycosis.

### BACKGROUND ART

Onychomycosis is an infection of the nail mainly caused by causative pathogens such as Trichophyton rubrum or Trichophyton mentagrophytes, which are classified into dermatophytes. This disease has been known as “athlete's foot of the nails.” If a person is affected with onychomycosis, in addition to changes in the appearance, such as deformation of the nail, it becomes difficult for the person to walk due to thickening of nails. Thus, physical and/or mental burden is imposed on the patient. In addition, if onychomycosis is left without being treated, it may result in spread of the infection.

As therapeutic agents for onychomycosis, internal medicines and external medicines applied to nails have been known. As an external medicine applied to onychomycosis, a liquid agent for external use has been approved and sold for the first time in 2014 in Japan, and the market has been expanding. This liquid agent for external use is a preparation comprising efinaconazole as an active ingredient. Efinaconazole has low affinity with keratin which is a component constituting the nail, and high permeability through the nail. Accordingly, it is said that efinaconazole is highly useful as an external medicine for nails (Non Patent Literature 1). However, the existing efinaconazole-containing liquid agent for external use still has problems specific to liquid agents for external use, such that, upon the application thereof, the agent liquid may be dropped even on the skin and may cause skin irritation, or such that the applied agent liquid may be exposed to non-application sites.

As a dosage form that solves the aforementioned problems of liquid agents for external use, a semi-solid preparation, the surface of which is solidified (film formation) after the application thereof (hereinafter referred to as “film-forming preparation”), has been known (Patent Literatures 1 to 6). In such a film-forming preparation, a volatile solvent contained in the preparation is evaporated after the application thereof, so that a non-volatile component such as a solidifying agent forms a solidified layer on the applied surface.

Moreover, an external-use antifungal agent for nails, comprising neticonazole or a salt thereof and a basic substance, has been known (Patent Literature 7). Example 2 of Patent Literature 7 discloses a nail lacquer comprising neticonazole hydrochloride, ethyl cellulose, diethyl sebacate, polyoxyethylene (9) lauryl ether, diisopropanolamine, dibutylhydroxytoluene, acetone and ethanol.

### PRIOR ART LITERATURES

### Patent Literatures

Patent Literature 1: JP Patent Publication (Kohyo) No. 2008-501805 A

Patent Literature 2: JP Patent Publication (Kohyo) No. 2009-519958 A

Patent Literature 3: JP Patent Publication (Kohyo) No. 2015-521996 A

Patent Literature 4: WO2014/104149

Patent Literature 5: JP Patent Publication (Kokai) No. 2016-27036 A

Patent Literature 6: WO2003/105903

Patent Literature 7: JP Patent Publication (Kokai) No. 2004-83439 A

### Non Patent Literatures

Non Patent Literature 1: Folia Pharmacologica Japonica 145, 250-258 (2015)

### SUMMARY OF INVENTION

### Object to be Solved by the Invention

By the way, the film-forming preparation is required to have properties such as “a short time required until film formation” and “sufficient preparation cohesiveness (peeling ability) after completion of the solidification, so that the film can be easily peeled (can be removed) without breaking the film.” Moreover, if the surface of the film-forming preparation is sticky after completion of the solidification, it causes problems such that the preparation is easily dropped when it comes into contact with non-application sites. Besides, in the aforementioned patent literatures, the stickiness on the surface after solidification is not explained at all.

Hence, it is an object of the present invention to provide an efinaconazole-containing film-forming preparation, which has no sticky surface after solidification thereof, has sufficient cohesiveness after solidification thereof, and is easily peeled without washing.

### Means for Solving the Object

As a result of intensive studies directed toward achieving the aforementioned object, the present inventors have revealed that an efinaconazole-containing film-forming preparation, which has no sticky surface after solidification thereof, has sufficient cohesiveness after solidification thereof, and is easily peeled without washing, can be realized by adding a surfactant to an ethyl cellulose base. In general, a surfactant as a pharmaceutical additive is used as a drug absorption promoter. However, surprisingly, the present inventors have found that when a surfactant is added to an efinaconazole-containing preparation comprising ethyl cellulose as a base, stickiness is not generated on the surface of the preparation after formation of a film, and thus, a peelable preparation having good cohesiveness can be obtained, thereby completing the present invention. Specifically, the gist of the present invention is as follows.
(1) A therapeutic preparation for onychomycosis, comprising:
   a) efinaconazole or a salt thereof,
   b) 1% by weight or more and 30% by weight or less of ethyl cellulose,
   c) 20% by weight or more of a volatile solvent, wherein the volatile solvent is water or a liquid component having a boiling point lower than a boiling point of water, and d) 0.01% by weight or more and less than 30% by weight of a surfactant.
(2) The preparation according to (1), wherein an amount of the surfactant in the preparation is 0.1% by weight or more and 25% by weight or less.
(3) The preparation according to (1), wherein an amount of the surfactant in the preparation is 1% by weight or more and 20% by weight or less.
(4) The preparation according to any one of (1) to (3), wherein an amount of the ethyl cellulose in the preparation is 5% by weight or more and 25% by weight or less.
(5) The preparation according to any one of (1) to (3), wherein an amount of the ethyl cellulose in the preparation is 10% by weight or more and 20% by weight or less.
(6) The preparation according to any one of (1) to (5), wherein an amount of the efinaconazole or a salt thereof in the preparation is 0.1% by weight or more and 30% by weight or less.
(7) The preparation according to any one of (1) to (5), wherein an amount of the efinaconazole or a salt thereof in the preparation is 0.5% by weight or more and 25% by weight or less.
(8) The preparation according to any one of (1) to (5), wherein an amount of the efinaconazole or a salt thereof in the preparation is 1% by weight or more and 20% by weight or less.
(9) The preparation according to any one of (1) to (8), wherein the surfactant comprises a cationic surfactant.
(10) The preparation according to (9), wherein the cationic surfactant is N-coconut oil fatty acid acyl-L-arginine-ethyl DL-pyrrolidone carboxylate.
(11) The preparation according to any one of (1) to (10), wherein the surfactant comprises an anionic surfactant.
(12) The preparation according to any one of (1) to (11), wherein the surfactant comprises an amphoteric surfactant.
(13) The preparation according to any one of (1) to (12), wherein the surfactant comprises a nonionic surfactant.
(14) The preparation according to any one of (1) to (13), wherein the surfactant has a glycerin backbone or a propylene glycol backbone.
(15) The preparation according to any one of (1) to (14), wherein the surfactant has a sorbitan backbone.
(16) The preparation according to any one of (1) to (15), wherein the volatile solvent comprises at least one selected from the group consisting of ethanol, isopropanol, ethyl acetate, acetone, diethyl ether, dimethyl ether, and water.
(17) The preparation according to any one of (1) to (16), wherein the volatile solvent comprises at least one volatile solvent other than water.
(18) The preparation according to (17), wherein an amount of the at least one volatile solvent other than water in the preparation is 50% by weight or more and 90% by weight or less.
(19) The preparation according to any one of (1) to (18), further comprising a non-volatile component other than the surfactant.
(20) The preparation according to (19), wherein an amount of the non-volatile component other than the surfactant in the preparation is 7% by weight or less.
(21) The preparation according to (1 9) or (20), wherein the non-volatile component other than the surfactant comprises an ester.
(22) The preparation according to (19) or (20), wherein the non-volatile component other than the surfactant comprises an alcohol.
(23) The preparation according to any one of (1) to (22), which is a liquid agent for external use, and is a film forming agent that solidifies after it has been applied to an effected area.
(24) A preparation comprising:
   a) a pharmaceutically active ingredient,
   b) ethyl cellulose,
   c) 20% by weight or more of a volatile solvent, wherein the volatile solvent is water or a liquid component having a boiling point lower than a boiling point of water, and
   d) a cationic surfactant.
(25) The preparation according to (24), wherein the cationic surfactant is N-coconut oil fatty acid acyl-L-arginine-ethyl DL-pyrrolidone carboxylate.

### Advantageous Effects of Invention

According to the present invention, an efinaconazole-containing film-forming preparation, which has no sticky surface after solidification thereof, has sufficient cohesiveness after solidification thereof, and can be easily peeled without washing, can be provided.

### EMBODIMENTS FOR CARRYING OUT THE INVENTION

Hereinafter, the present invention will be described in detail. The storage form of the preparation of the present invention is not particularly limited, and the preparation is preferably stored in a tube or an ointment jar.

The preparation of the present invention comprises (a) efinaconazole or a salt thereof as a drug, and further, at least, (b) ethyl cellulose, (c) a volatile solvent, and (d) a surfactant. The preparation of the present invention is preferably a liquid agent for external use, and also a film-forming agent that solidifies after it has been applied to an affected area.

### (a) Efinaconazole or a Salt Thereof

Efinaconazole that can be used in the present invention may be either free-form efinaconazole or a pharmaceutically acceptable salt of efinaconazole. Efinaconazole is also referred to as KP-103 or (2R,3R)-2-(2,4-difluorophenyl)-3-(4-methylenepiperidin-l-yl)-1-(1H-1,2,4-triazol-1-yl)but an-2-ol. Herein, the pharmaceutically acceptable salt of efinaconazole is not particularly limited, and it may be either an inorganic salt or an organic salt. Examples of the inorganic salt of efinaconazole include hydrochloride, hydrobromide, nitrate, sulfate, and phosphate. Examples of the organic salt of efinaconazole include formate, acetate, trifluoroacetate, propionate, lactate, tartrate, oxalate, fumarate, maleate, citrate, malonate, methane sulfonate, and toluene sulfonate. From the viewpoint of availability, free-form efinaconazole is preferably used.

As such efinaconazole or a salt thereof, efinaconazole or a salt thereof produced according to a known method can be used, or a commercially available product satisfying the above-described properties can also be used. In addition, efinaconazole is commercially available, and it can be purchased, for example, from Sigma-Aldrich Japan (Product code: SML1244), etc.

In order to obtain sufficient medicinal efficacy, the content of efinaconazole is preferably 0.1% by weight or more, more preferably 0.5% by weight or more, further preferably 1% by weight or more, still further preferably 2% by weight or more, particularly preferably 5% by weight or more, and most preferably 7% by weight or more, with respect to the total amount of the components of the preparation. On the other hand, from the viewpoint of ensuring viscosity easing the application of a preparation, the upper limit of the content of efinaconazole is preferably 30% by weight or less, more preferably 25% by weight or less, further preferably 20% by weight or less, particularly preferably 15% by weight or less, and most preferably 10% by weight or less.

### (b) Ethyl Cellulose

Ethyl cellulose that can be used in the preparation of the present invention is not particularly limited, as long as it may be ethyl cellulose, in which the hydrogen atoms (H) of hydroxyl groups (OH group) of cellulose are substituted with ethyl groups (C₂H₅). In the present description, the percentage at which the hydrogen atoms of hydroxyl groups in ethyl cellulose are substituted with ethyl groups is referred to as “ethyl group substitution percentage.”

The above-described ethyl group substitution percentage is not particularly limited, and it may be 10% or more, or 20% or more, or 30% or more, or 40% or more, or 45% or more. In addition, the ethyl group substitution percentage may be 100%, or 90% or less, or 80% or less, or 70% or less, or 60% or less, or 55% or less. From the viewpoint of solubility in a volatile solvent, etc., the ethyl group substitution percentage is preferably 30% or more and 70% or less, more preferably 40% or more and 60% or less, and further preferably 45% or more and 55% or less.

The weight average molecular weight (MW) of the above-described ethyl cellulose is not particularly limited, and it may be 20000 or more, or 40000 or more, or 60000 or more. On the other hand, it may be 400000 or less, or 300000 or less, or 230000 or less. In the present invention, a preparation having viscosity that eases the application thereof can be obtained by appropriately selecting the weight average molecular weight of ethyl cellulose and the content of ethyl cellulose with respect to the total amount of the components of the preparation.

Ethyl cellulose used in the present invention may be one type, or two or more types of ethyl cellulose, in which the ethyl group substitution percentage or the weight average molecular weight is different from one another, may be used in combination.

In a certain example of the present invention, there may be a case where ethyl cellulose is not dissolved in a volatile solvent if the ethyl group substitution percentage in ethyl cellulose is low. This may cause gelatinization or the like. On the other hand, in another example of the present invention, there may be a case where ethyl cellulose is not dissolved in a volatile solvent if the ethyl group substitution percentage is high.

In one example of the present invention, there may be a case where a preparation does not have sufficient viscosity and thus, it becomes difficult to apply the preparation, if the weight average molecular weight of ethyl cellulose is low. In another example of the present invention, there may be a case where a preparation becomes hard and thus, it becomes difficult to apply the preparation, if the weight average molecular weight of ethyl cellulose is high.

Ethyl cellulose produced by a known method can be used, or a commercially available product satisfying the above-described properties can also be used. Moreover, ethyl cellulose is commercially available, and examples of the commercially available ethyl cellulose include: “ETHOCEL STD4,” “ETHOCEL STD7,” “ETHOCEL STD10,” “ETHOCEL STD20,” “ETHOCEL STD45,” “ETHOCEL STD100,” “ETHOCEL STD200,” “ETHOCEL STD300,” “ETHOCEL MED50,” and “ETHOCEL MED70,” which are manufactured by The Dow Chemical; and “T10 Pharm,” “N7 Pharm,” “N10 Pharm,” “N14 Pharm,” “N22 Pharm,” “N50 Pharm,” and “N100 Pharm,” which are manufactured by Ashland.

The content of ethyl cellulose with respect to the total amount of the components of the preparation is not particularly limited, as long as it is within the range that does not impair the applicability of the preparation as a coating agent. In one example of the present invention, the lower limit of the content of ethyl cellulose is preferably 1% by weight or more, more preferably 5% by weight or more, and particularly preferably 10% by weight or more, with respect to the total amount of the components of the preparation. If the content of ethyl cellulose is too high, the preparation becomes hard and thus it becomes difficult to apply the preparation. The upper limit of the content ethyl cellulose is preferably 30% by weight or less, more preferably 25% by weight or less, and most preferably 20% by weight or less, with respect to the total amount of the components of the preparation.

### (c) Volatile Solvent

The volatile solvent that can be used in the present invention is water or a liquid component having a boiling point lower than a boiling point of water. Examples of the volatile solvent include water, ethanol, isopropanol, ethyl acetate, and acetone. The volatile solvent is particularly preferably ethanol.

If the content of the volatile solvent used in the present invention is too low, the preparation becomes hard. On the other hand, if the content of the volatile solvent is too high, it is dropped upon the application thereof, and thus, it becomes difficult to apply the preparation. The content of the volatile solvent is not particularly limited, as long as it is within the range that does not impair the applicability of the preparation. The lower limit of the content of the volatile solvent is preferably 35% by weight or more, more preferably 40% by weight or more, further preferably 50% by weight or more, and particularly preferably 60% by weight or more, with respect to the total amount of the components of the preparation. The upper limit of the content of the volatile solvent is preferably 90% by weight or less, more preferably 85% by weight or less, further preferably 80% by weight or less, and particularly preferably 75% by weight or less, with respect to the total amount of the components of the preparation.

The volatile solvent may be used alone, or may be used in the form of a mixture of two or more types.

The volatile solvent preferably comprises at least one type of volatile solvent other than water.

The content of the volatile solvent other than water in the preparation of the present invention is preferably 50% by weight or more and 90% by weight or less, and more preferably 60% by weight or more and 80% by weight or less. The content of the volatile solvent other than water is further preferably 60% by weight or more and 75% by mass or less.

### (d) Surfactant

The surfactant described in the present description is not particularly limited, as long as it has a hydrophilic group and a hydrophobic group in a single molecule thereof, or it forms a micelle in a solvent. Examples of the surfactant include substances listed as surfactants in the website of Japan Surfactant Industry Association (http://www.jp-surfactant.jp/index.html), and substances classified into surfactants in Encyclopedia of Pharmaceutical Additives 2016 (edited by International Pharmaceutical Excipients Council Japan). The surfactants can be classified into a nonionic surfactant, an anionic surfactant, a cationic surfactant, and an amphoteric surfactant. Otherwise, the surfactants can also be classified into surfactants having a glycerin backbone or a propylene glycol backbone, and surfactants having a sorbitan backbone.

Examples of the nonionic surfactant include sorbitan monooleate, sorbitan monostearate, sorbitan tristearate, sorbitan monolaurate, sorbitan monopalmitate, sorbitan sesquioleate, sorbitan trioleate, glyceryl monooleate, glyceryl monostearate, glyceryl monomyristate, diglyceryl monooleate, decaglyceryl laurate, hexaglyceryl polyricinoleate, decaglyceryl pentastearate, α-monoisostearyl glyceryl ether, diglyceryl monoisostearate, diglyceryl monostearate, polyoxyethylene glycerin monostearate, polyoxyethylene glyceryl triisostearate, polyoxyethylene coconut oil fatty acid glyceryl, propylene glycol monostearate, ethylene glycol monostearate, polyoxyethylene (9) lauryl ether, polyoxyethylene (2) lauryl ether, polyoxyethylene (4,2) lauryl ether, polyoxyethylene (5) nonyl phenyl ether, polyoxyethylene (7,5) nonyl phenyl ether, polyoxyethylene (10) nonyl phenyl ether, polyoxyethylene (3) octyl phenyl ether, polyoxyethylene (10) octyl phenyl ether, polyoxyethylene oleyl ether, polyoxyethylene behenyl ether, polyoxyethylene stearyl ether, polyoxyethylene cetyl ether, polyoxyethylene cetostearyl ether, polyethylene glycol distearate, polyethylene glycol monostearate, polyoxyethylene lanoline, polyoxyethylene sorbitan monolaurate, polyoxyethylene sorbitan monostearate, polyoxyethylene sorbitan trioleate, polyoxyethylene sorbitan tetraoleate, polyoxyethylene sorbit beeswax, polyoxyethylene (10) oleylamine, polyoxy (5) oleylamine, polyoxy (5) oleamide, polyoxyethylene (2) monolaurate, polyoxyethylene castor oil (hydrogenated castor oil), polyoxyl stearate 40, Macrogol (registered trademark) 400, Cetomacrogol 1000, Lauromacrogol, polyoxyethylene (20) polyoxypropylene (20) glycol, polyoxyethylene (10) polyoxypropylene (4) cetyl ether, Polysorbate 20, Polysorbate 40, Polysorbate 60, Polysorbate 65, Polysorbate 80, polyethylene glycol monooleate, polyethylene glycol monolaurate, polyethylene glycol monostearate, diethanolamide laurate, hydrogenated soybean phospholipid, surfactin, dimethyl polysiloxane, cyclomethicone, methylphenyl polysiloxane, and lauryl pyrrolidone.

Examples of the anionic surfactant include sodium lauryl sulfate, potassium lauryl sulfate, triethanolamine lauryl sulfate, sodium cetyl sulfate, sodium lauroyl sarcosinate, sodium di-2-ethylhexyl sulfosuccinate, polyoxyethylene (10) lauryl ether sodium phosphate, polyoxyethylene sodium lauryl ether sulfate, dicetyl phosphate, sodium lauryl phosphate, polyoxyethylene (4) sodium lauryl ether phosphate, polyoxyethylene (5) cetyl ether sodium phosphate, polyoxyethylene (6) oleyl ether sodium phosphate, polyoxyethylene stearyl ether sodium phosphate, nonyl phenyl polyoxyethylene ether sulfate ammonium salt, polyoxyethylene oleyl ether diethanolamine phosphate, sodium N-cocoyl-N-methylaminoethylsulfonate (cocoyl methyl taurine sodium), sodium dodecylbenzenesulfonate, sodium N-acyl-L-glutamate, and surfactin sodium.

Examples of the cationic surfactant include stearyl trimethyl ammonium chloride, distearyl dimethyl ammonium chloride, benzalkonium chloride, stearyl dimethyl benzyl ammonium chloride, and N-cocoyl-L-arginine ethyl ether DL-pyrrolidone carboxylate (N-coconut oil fatty acid acyl-L-arginine-ethyl DL-pyrrolidone carboxylate).

Examples of the amphoteric surfactant include lauryldimethylaminoacetic acid betaine, 2-alkyl-N-carboxymethyl-N-hydroxyethyl imidazolinium betaine, disodium cocoamphodiacetate, lauric acid amidopropyl betaine, 2-[(3-dodecaneamidopropan-1-yl)dimethylammonio]acetate, and lauryl dimethylamine oxide.

Examples of the surfactant having a glycerin backbone include glyceryl monooleate, glyceryl monostearate, glyceryl monomyristate, diglyceryl monooleate, decaglyceryl laurate, hexaglyceryl polyricinoleate, decaglyceryl pentastearate, α-monoisostearyl glyceryl ether, diglyceryl monoisostearate, diglyceryl monostearate, polyoxyethylene glyceryl monostearate, polyoxyethylene glyceryl triisostearatc, and polyoxyethylene coconut oil fatty acid glyceryl.

An example of the surfactant having a propylene glycol backbone is propylene glycol monostearate.

Examples of the surfactant having a sorbitan backbone include sorbitan monooleate, sorbitan monostearate, sorbitan tristearate, sorbitan monolaurate, sorbitan monopalmitate, sorbitan sesquioleate, sorbitan trioleate, polyoxyethylene sorbitan monolaurate, polyoxyethylene sorbitan monostearate, polyoxyethylene sorbitan trioleate, polyoxyethylene sorbitan tetraoleate, polyoxyethylene sorbit beeswax, Polysorbate 20, Polysorbate 40, Polysorbate 60, Polysorbate 65, and Polysorbate 80.

The above-classified surfactants may be used alone, or may also be used in the form of a mixture of two or more types.

The surfactant is preferably a cationic surfactant, and is particularly preferably N-coconut oil fatty acid acyl-L-arginine-ethyl DL-pyrrolidone carboxylate. A keratin protein which is a component constituting the nail is rich in negative charges (anions). Thus, it is assumed that a cationic surfactant interacts with the negative charges of keratin to destroy the conformation of the protein, thereby enhancing the permeability of a drug.

When the content of the surfactant is too high with respect to the total amount of the components of the preparation, the adhesiveness of the preparation to nails is reduced. The surfactant may be added to the preparation within a range that does not impair applicability or adhesiveness to nails. The content of the surfactant is preferably 0.01% by weight or more and less than 30% by weight, more preferably 0.1% by weight or more and 25% by weight or less, further preferably 1% by weight or more and 20% by weight or less, particularly preferably 1% by weight or more and 15% by weight or less, and most preferably 1% by weight or more and 10% by weight or less, with respect to the total amount of the components of the preparation.

### (e) Other Components

The preparation of the present invention may comprise non-volatile components such as (e1) esters, (e2) alcohols, (e3) a nitrogen-containing compound, (e4) organic acid, and (e5) terpenes, alone or in combination, as necessary. Besides, the term “non-volatile component” is used in the present description to mean a liquid or solid compound having a boiling point higher than a boiling point of water.

### (e1) Esters

Examples of the esters include: fatty acid monovalent alcohol esters, such as isopropyl isostearate, methyl stearate, butyl stearate, ethyl linoleate, isopropyl linoleate, ethyl oleate, oleyl oleate, decyl oleate, isopropyl myristate, butyl myristate, myristyl myristate, cetyl isooctanoate, octyldodecyl myristate, diethyl adipate, diisopropyl adipate, diisobutyl adipate, methyl laurate, hexyl laurate, methyl myristate, methyl caproate, methyl palmitate, cetyl palmitate, retinol palmitate, isopropyl palmitate, isostearyl palmitate, isopropyl myristate, diethyl sebacate, diisopropyl sebacate, cetyl 2-ethylhexanoate, hexadecyl isostearate, butyl acetate, or benzyl acetate; aromatic carboxylic acid monovalent alcohol esters, such as diethyl phthalate, dibutyl phthalate, or triethyl citrate; lactic acid esters, such as ethyl lactate, cetyl lactate, or myristyl lactate; carbonic acid esters, such as ethylene carbonate or propylene carbonate; and triacyl glycerol esters, such as triacetin, tricaprylin, a tri(caprylic-capric acid) glyceride-tristearate glyceride mixture, or tri(caprylic-capric acid)glycerin.

These esters may be used alone or in the form of a mixture of two or more types.

Such esters may be added to the preparation of the present invention within the range in which the preparation is easily applicable and does not cause stickiness on the surface. Among others, the amount of the esters is preferably 20% by weight or less, more preferably 15% by weight or less, further preferably 10% by weight or less, and particularly preferably 7% by weight or less, with respect to the total amount of the components of the preparation.

When the esters are used, the esters may be replaced with a part of the volatile solvent constituting the preparation, so that they may be added to the preparation.

### (e2) Alcohols

Examples of the alcohols include fatty acid alcohols, such as isostearyl alcohol, lauryl alcohol, 2-octyldodecanol, 2-hexyldecanol, cetanol, myristyl alcohol, oleyl alcohol, ethylene glycol, triethylene glycol, propylene glycol, dipropylene glycol, 1,3-butanediol, 1,2,6-hexanetriol, 2-ethyl-1,3-hexanediol, or glycerin; and aromatic alcohols, such as glycol salicylate or benzyl alcohol.

These alcohols may be used alone or in the form of a mixture of two or more types. Such alcohols may be added to the preparation within the range in which the preparation is easily applicable and does not cause stickiness on the surface. Among others, the amount of the alcohols is preferably 20% by weight or less, more preferably 15% by weight or less, further preferably 10% by weight or less, and particularly preferably 7% by weight or less, with respect to the total amount of the components of the preparation.

When the alcohols are used, the alcohols may be replaced with a part of the volatile solvent constituting the preparation, so that they may be added to the preparation.

### (e3) Nitrogen-Containing Compound

Examples of the nitrogen-containing compound include: aliphatic amines such as ethylenediamine, diisopropanolamine, diethanolamine, triisopropanolamine, or triethanolamine; amides such as N-methyl-2-pyrrolidone, crotamiton, or urea; and amino acids such as cysteine or methionine.

These nitrogen-containing compounds may be used alone or in the form of a mixture of two or more types. Such nitrogen-containing compounds may be added to the preparation within the range in which the preparation is easily applicable and does not cause stickiness on the surface. Among others, the amount of the nitrogen-containing compounds is preferably 20% by weight or less, more preferably 15% by weight or less, further preferably 10% by weight or less, and particularly preferably 7% by weight or less, with respect to the total amount of the components of the preparation.

When the nitrogen-containing compounds are used, the nitrogen-containing compounds may be replaced with a part of the volatile solvent constituting the preparation, so that they may be added to the preparation.

### (e4) Organic Acid

Examples of the organic acid include: fatty acids, such as adipic acid, isostearic acid, caprylic acid, capric acid, acetic acid, stearic acid, lactic acid, palmitic acid, propionic acid, fumaric acid, behenic acid, maleic acid, or myristic acid; and aromatic carboxylic acids, such as benzoic acid, phthalic acid, or salicylic acid.

These organic acids may be used alone or in the form of a mixture of two or more types. Such organic acids may be added to the preparation within the range in which the preparation is easily applicable and does not cause stickiness on the surface. Among others, the amount of the organic acids is preferably 20% by weight or less, more preferably 15% by weight or less, further preferably 10% by weight or less, and particularly preferably 7% by weight or less, with respect to the total amount of the components of the preparation.

When the organic acids are used, the organic acids may be replaced with a part of the volatile solvent constituting the preparation, so that they may be added to the preparation.

### (e5) Terpenes

Examples of the terpenes include squalane, squalene, menthol, and borncol.

These terpenes may be used alone or in the form of a mixture of two or more types. Such terpenes may be added to the preparation within the range in which the preparation is easily applicable and does not cause stickiness on the surface. Among others, the amount of the terpenes is preferably 20% by weight or less, more preferably 15% by weight or less, further preferably 10% by weight or less, and particularly preferably 7% by weight or less, with respect to the total amount of the components of the preparation.

When the terpenes are used, the terpenes may be replaced with a part of the volatile solvent constituting the preparation, so that they may be added to the preparation.

The preparation according to the present invention may further comprise various types of known additives, such as a stabilizer, a perfume, a coloring agent, and a preservative, within the range that does not impair the effects of the present invention.

### [Preparation Comprising Cationic Surfactant]

The present invention further relates to a preparation, comprising:

a) a pharmaceutically active ingredient,

b) ethyl cellulose,

c) 20% by weight or more of a volatile solvent, wherein the volatile solvent is water or a liquid component having a boiling point lower than a boiling point of water, and

d) a cationic surfactant.

The pharmaceutically active ingredient that can be used in the present invention is not particularly limited. Examples of the pharmaceutically active ingredient include antifungal agents, such as efinaconazole, amorolfine hydrochloride, terbinafine hydrochloride, miconazole nitrate, isoconazole nitrate, sulconazole nitrate, oxiconazole nitrate, econazole nitrate, cloconazole nitrate, neticonazole nitrate, tolnaftate, tolciclate, liranaftate, clotrimazole, tioconazole, bifonazole, ketoconazole, luliconazole, siccanin, pyrrolnitrin, griseofulvin, undecylenic acid, fluconazole, itraconazole, naftifine, ciclopirox olamine, and exalamide. When the above-described pharmaceutically active ingredient is a free-form pharmaceutically active ingredient, its salt may also be used. When the above-described pharmaceutically active ingredient is a salt, the free-form may be used.

In order to obtain sufficient medicinal efficacy, the content of the pharmaceutically active ingredient is preferably 0.1% by weight or more, more preferably 0.5% by weight or more, further preferably 1% by weight or more, still further preferably 2% by weight or more, particularly preferably 5% by weight or more, and most preferably 7% by weight or more, with respect to the total amount of the components of the preparation. On the other hand, from the viewpoint of ensuring the viscosity easing the application of a preparation, the upper limit of the content of the pharmaceutically active ingredient is preferably 30% by weight or less, more preferably 25% by weight or less, further preferably 20% by weight or less, particularly preferably 15% by weight or less, and most preferably 10% by weight or less.

The ethyl cellulose and the volatile solvent are as described above in the present description.

In order to obtain the effect of improving the stability of the preparation, the content of the cationic surfactant is preferably 0.01% by weight or more and less than 30% by weight, more preferably 0.1% by weight or more and 25% by weight or less, further preferably 1% by weight or more and 20% by weight or less, particularly preferably 1% by weight or more and 15% by weight or less, and most preferably 1% by weight or more and 10% by weight or less, with respect to the total amount of the components of the preparation.

The preparation comprising a cationic surfactant may comprise various types of components described in the present description.

As a cationic surfactant, N-coconut oil fatty acid acyl-L-arginine-ethyl DL-pyrrolidone carboxylate is particularly preferable.

### [Vessel]

The vessel used in the present invention is not particularly limited, and a commonly used vessel can be used. Examples of the vessel used herein include an ointment jar made from polypropylene and an aluminum tube.

### [Method of Preparing Pharmaceutical Composition Used in Production of Preparation]

The preparation of the present invention can be prepared using the below-mentioned pharmaceutical composition. The pharmaceutical composition can be prepared by mixing and stirring the following components. That is to say, (a) efinaconazole or a salt thereof, (b) ethyl cellulose, (c) a volatile solvent, and (d) a surfactant are mixed and stirred to obtain a pharmaceutical composition. With regard to the order of mixing these components, for example, the component (a) to the component (d) may be successively added, or the component (b) and the component (c) may have previously been mixed with each other, and the component (a) and the component (d) may be then mixed with the obtained mixture. Individual components may be prepared in a vessel for storing a preparation, or the components may be prepared in each different vessels and may be then transferred into a storage vessel.

Upon the mixing and stirring of the components, the components may be heated, so as to facilitate the uniform mixing of the components. The heating temperature is preferably 30° C. or higher, more preferably 40° C. or higher, and most preferably 50° C. or higher. In order to prevent deterioration or volatilization of the components of the preparation composition, the upper limit of the temperature is preferably 100° C. or lower, and more preferably 90° C. or lower.

In the above-described heating temperature range, the heating temperature is preferably 30° C. or higher and 100° C. or lower, and more preferably 40° C. or higher and 90° C. or lower.

### [Method for Producing Preparation]

As a method for preparing the preparation of the present invention, a method of filling a pharmaceutical composition in a storage vessel can be preferably applied.

### [Intended Use of Preparation]

The preparation of the present invention can be used as an agent for coating nails.

Since the coating agent of the present invention comprises efinaconazole or a salt thereof, it can be used as a therapeutic agent for onychomycosis.

The thickness of the present preparation applied onto the nail can be adjusted within a range that does not impair the effects of the therapeutic agent for onychomycosis or the ease of use as an agent for coating nails. The thickness of the preparation upon the application thereof may be 3 mm or less, or 2 mm or less, or 1 mm or less, or may also be 20 μm or more, or 50 μm or more, or 100 μm or more.

When the preparation of the present invention is applied onto the nail, it has appropriate adhesive strength. The appropriate adhesive strength means adhesive strength to such an extent that it is not peeled off, even if the surface of the nail, at least, after application of the preparation thereto, is turned to the ground.

When the preparation of the present invention is applied onto the nail, it has appropriate viscosity. The appropriate viscosity means viscosity to such an extent that the dropping of the preparation can hardly be confirmed by naked eyes, even if the surface of the nail, at least, immediately after application of the preparation thereto, is turned vertical to the ground.

When the thus applied preparation is removed, the preparation may be picked with fingers or tweezers and may be then peeled. When the preparation is removed after the application thereof, from the viewpoint of drying even the inner portion of the applied preparation that is not contacted with the air, and also, from the viewpoint of sufficiently delivering the active ingredient into the nail, the removing timing is preferably 1 hour or more after the application of the preparation, more preferably two hours or more after the application thereof, further preferably 6 hours or more after the application thereof, particularly preferably 12 hours or more after the application thereof, and most preferably 24 hours or more after the application thereof.

The preparation of the present invention does not have stickiness on the surface of the preparation after solidification thereof. On the other hand, the present preparation has sufficient adhesive strength to the applied surface, and thus, the preparation is not easily dropped from the applied surface, before the peeling operation is carried out.

### EXAMPLES

Hereinafter, the present invention will be more specifically described in Examples, Test Examples, and Preparation Examples. However, these examples are not intended to limit the scope of the present invention. It is to be noted that the numerical value of each component shown in Tables 1 to 5 and Table 7 indicates part by mass. The numerical value of each component shown in Table 8 indicates % by mass.

### (Evaluation Method) Film Formation Time

From the following viewpoints, the time required for the formation of a film from the preparation was evaluated.
∘: After application of the preparation, the time required until solidification of the surface was less than 10 minutes.
x: After application of the preparation, the time required until solidification of the surface was 10 minutes or more.

### (Evaluation Method) Peeling Ability

From the following viewpoints, the peeling ability of the preparation was evaluated.
α2: When the preparation was peeled off, it was peeled off as an aggregate, without particular disintegration.
x: When the preparation was peeled off, the preparation remained on the applied surface and was disintegrated.

### (Evaluation Method) Stickiness on the Surface

From the following viewpoints, the stickiness of the preparation was evaluated.
∘: Sixty minutes after application of the preparation, stickiness was not felt, even touching the surface of the preparation.
Δ: Sixty minutes after application of the preparation, slight stickiness was felt, when touching the surface of the preparation.
x: Sixty minutes after application of the preparation, stickiness was felt, when touching the surface of the preparation.

### (Evaluation Method) Evaluation of Permeability Through Human Nails

A dead human nail was immersed in an infiltrate (e.g., a 5% ethanol-containing 0.01 mol/L phosphate buffered saline (pH 7.2 to 7.4)) for 30 minutes or more, and thereafter, the nail was cut into a section with a diameter of approximately 5 to 6 mm. The nail plate side of the cut nail was attached along a rubber-made O-ring with an inner diameter of 4 mm, and the gap was then caulked with an adhesive. The upper and lower sides thereof were sandwiches between the processed plastic-made screw vial and screw cap, and were then equipped into an automatic sampling apparatus (manufactured by CosMED Pharmaceutical Co. Ltd.; TransView C12). As a receptor solution, a 5% ethanol-containing 0.01 mol/L phosphate buffered saline (pH 7.2 to 7.4) was used, and the receptor temperature was set at 32° C. Twenty-four hours after initiation of the test, an aliquot of the receptor solution was sampled, and the amount of efinaconazole permeating through the human nail in the receptor solution was then quantified by LC/MS/MS. As a control preparation, Clenafin (registered trademark) (manufactured by Kaken Pharmaceutical Co., Ltd.; 10% Efinaconazole Solution Preparation) was used. [0078]

### Example 1

0.32 g of Ethyl cellulose (ethyl group substitution percentage: 48.0% to 49.5%; weight average molecular weight: 180000), 1.28 g of ethanol, 0.20 g of efinaconazole, and 0.20 g of AMPHITOL (registered trademark) 20AB (20%-30% lauric acid amidopropyl betaine solution) were successively added into a polypropylene-made ointment jar with a volume of 6 mL, and they were then mixed and stirred using a stirring degassing machine at 2000 rpm for 15 minutes. Thereafter, the resulting mixture was degassed at 2200 rpm for 90 seconds, to obtain a homogeneous pharmaceutical composition. The components of the preparation and the weight percentage (%) thereof are shown in Table 1.

### Example 2

A preparation was produced by the same method as that of Example 1, with the exception that efinaconazole, ethyl cellulose, ethanol, and surfactin sodium were mixed with one another at the ratio shown in Table 1.

### Comparative Examples 1 to 4

In the case of a film-forming preparation, in order to ensure the cohesiveness (peeling ability) of the preparation after completion of the film formation, a predetermined amount of non-volatile plasticizer is added in many cases. As common plasticizers, esters and alcohols have been known.

A preparation was produced by the same method as that of Example 1, with the exception that efinaconazole, ethyl cellulose, ethanol, and ester or alcohol were mixed with one another at the ratio shown in Table 1.

### (Ethyl Cellulose)

Ethyl cellulose A: ethyl group substitution percentage: 48.0%-49.5%; weight average molecular weight: 180000

Besides, since the ethyl group substitution percentage and the weight average molecular weight may be changed depending on the production lot of ethyl cellulose used, the values described in the present description are merely representative values, and thus, are not necessarily limited thereto.

TABLE 1

| | | Ex. 1 | Ex. 2 | Comp. Ex. 1 | Comp. Ex. 2 | Comp. Ex. 3 | Comp. Ex. 4 |
|---|---|---|---|---|---|---|---|
| Drug substance | Efinaconazole | 10 | 10 | 10 | 10 | 10 | 10 |
| Base | Ethyl cellulose A | 16 | 16 | 16 | 16 | 16 | 16 |
| Volatile solvent | Ethanol | 64 | 69 | 74 | 64 | 64 | 64 |
| Surfactant | AMPHITOL 20AB | 10 | | | | | |
| | Surfactin sodium | | 5 | | | | |
| Alcohol | 2-Hexyl-1-decanol | | | | 10 | | |
| Ester | Triacetin | | | | | 10 | |
| | Diisopropyl adipate | | | | | | 10 |

| | | | | | | |
|---|---|---|---|---|---|---|
| Film formation time | ◯ | ◯ | ◯ | ◯ | ◯ | ◯ |
| Peeling ability | ◯ | ◯ | X | X | ◯ | X |
| Surface stickiness | ◯ | ◯ | ◯ | X | X | X |

Stickiness on the surface was not observed in both of the preparations of Examples 1 and 2, to which a surfactant had been added. In addition, in Examples 1 and 2, the preparation could be removed without performing a special operation of washing the preparation with water, a solvent or the like, and also, the preparation did not remain after the peeling. In Comparative Example 1, in which a non-volatile component other than the drug and the base had not been added to the preparation, the preparation was disintegrated upon the peeling thereof. In Comparative Examples 2 to 4, in which alcohols or esters had been added, instead of a surfactant, stickiness was observed on the surface of the preparation.

### Examples 3 to 12

A preparation was produced by the same method as that of Example 1, with the exception that efinaconazole, ethyl cellulose, ethanol, and a surfactant were mixed with one another at the ratio shown in Table 2.

TABLE 2

| | | Ex. 3 | Ex. 4 | Ex. 5 | Ex. 6 | Ex. 7 | Ex. 8 |
|---|---|---|---|---|---|---|---|
| Drug substance | Efinaconazole | 10 | 10 | 10 | 10 | 10 | 10 |
| Base | Ethyl cellulose A | 16 | 16 | 16 | 16 | 16 | 16 |
| Volatile solvent | Ethanol | 64 | 64 | 64 | 64 | 64 | 64 |
| Surfactant | Polyoxyethylene sorbitan | 10 | | | | | |
| | monooleate | | | | | | |
| | Diglyceryl monooleate | | 10 | | | | |
| | coconut oil fatty acid diethanol | | | 10 | | | |
| | amide solution | | | | | | |
| | Lauromacrogol | | | | 10 | | |
| | Polyoxyethylene coconut oil fatty | | | | | 10 | |
| | acid sorbitan | | | | | | |
| | Polyethylene glycol monooleate | | | | | | 10 |

| | | | | | | |
|---|---|---|---|---|---|---|
| Film formation time | ◯ | ◯ | ◯ | ◯ | ◯ | ◯ |
| Peeling ability | ◯ | ◯ | ◯ | ◯ | ◯ | ◯ |
| Surface stickiness | ◯ | ◯ | ◯ | ◯ | ◯ | ◯ |

| | | Ex. 9 | Ex. 10 | Ex. 11 | Ex. 12 |
|---|---|---|---|---|---|
| Drug substance | Efinaconazole | 10 | 10 | 10 | 10 |
| Base | Ethyl cellulose A | 16 | 16 | 16 | 16 |
| Volatilc solvent | Ethanol | 71 | 64 | 64 | 64 |
| Surfactant | N-coconut oil fatty acid | 3 | | | |
| | acyl-L-arginine ethyl | | | | |
| | DL-pyrrolidone carboxylate | | | | |
| | 30.5% Sodium lauroyl sarcosinate | | 10 | | |
| | 25% Sodium methyl cocoyl taurate | | | 10 | |
| | Polyoxyethylene lauryl ether sodium | | | | 10 |
| | phosphate | | | | |

| | | | | |
|---|---|---|---|---|
| Film formation time | ◯ | ◯ | ◯ | ◯ |
| Peeling ability | ◯ | ◯ | ◯ | ◯ |
| Surface stickiness | ◯ | ◯ | ◯ | ◯ |

With regard to all of the preparations in Examples 3 to 8, in which a nonionic surfactant had been added, in Example 9, in which a cationic surfactant had been added, and in Examples 10 to 12, in which an anionic surfactant had been added, no stickiness was observed on the surface of the preparation. Moreover, in Examples 3 to 12, the preparation could be removed without performing a special operation of washing the preparation with water, a solvent or the like, and also, the preparation did not remain after the peeling.

### Examples 13 to 17 and Comparative Example 5

A preparation was produced by the same method as that of Example 1, with the exception that efinaconazole, ethyl cellulose, ethanol, and a surfactant were mixed with one another at the ratio shown in Table 3.

TABLE 3

| | | Ex. 13 | Ex. 14 | Ex. 15 | Ex. 16 | Ex. 17 | Comp. Ex. 5 |
|---|---|---|---|---|---|---|---|
| Drug substance | Efinaconazole | 10 | 10 | 10 | 10 | 10 | 10 |
| Base | Ethyl cellulose A | 16 | 16 | 16 | 16 | 16 | 16 |
| Volatile solvent | Ethanol | 73 | 73 | 71 | 54 | 54 | 44 |
| Surfactant | Surfactin sodium | | | 3 | | | |
| | Polyoxyethylene coconut oil fatty | 1 | | | 20 | | 30 |
| | acid sorbitan | | | | | | |
| | Polyoxyethylene lauryl ether | | 1 | | | 20 | |
| | sodium phosphate | | | | | | |

| | | | | | | |
|---|---|---|---|---|---|---|
| Film formation time | ◯ | ◯ | ◯ | ◯ | ◯ | — |
| Peeling ability | ◯ | ◯ | ◯ | ◯ | ◯ | — |
| Surface stickiness | ◯ | ◯ | ◯ | ◯ | ◯ | — |

In all cases where the additive amounts of the surfactant were set at 1% by weight, 3% by weight and 20% by weight, with respect to the total amount of the components of the preparation (Example 13 to 17), stickiness was not observed on the surface of the preparation. Moreover, in Examples 13 to 17, the preparation could be removed without performing a special operation of washing the preparation with water, a solvent or the like, and also, the preparation did not remain after the peeling. When the surfactant was used in an additive amount of 30% by weight with respect to the total amount of the components of the preparation, ethyl cellulose was hardly dissolved upon the production thereof, and the adhesiveness of the preparation to the applied surface was decreased, and it became unusable as a preparation.

### Examples 18 and 19

A preparation comprising a surfactant and an ester serving as a non-volatile component was produced. A preparation was produced by the same method as that of Example 1, with the exception that efinaconazole, ethyl cellulose, ethanol, a surfactant, and an ester were mixed with one another at the ratio shown in Table 4.

TABLE 4

| | | Ex. 18 | Ex. 19 |
|---|---|---|---|
| Drug substance | Efinaconazole | 10 | 10 |
| Base | Ethyl cellulose A | 16 | 16 |
| Volatile solvent | Ethanol | 64 | 64 |
| Surfactant | Polyoxyethylene lauryl ether | 5 | 3 |
| | sodium phosphate | | |
| Ester | Triacetin | 5 | 7 |

| | | |
|---|---|---|
| Film formation time | ◯ | ◯ |
| Peeling ability | ◯ | ◯ |
| Surface stickiness | ◯ | ◯ |

In the case of the preparation containing a surfactant, even if an ester was comprised in the preparation in an amount of 5% by weight or 7% by weight with respect to the total amount of the components in the preparation (Example 18 and Example 19), a preparation having no stickiness on the surface could be obtained. In addition, in Examples 18 and 19, the preparation could be removed without performing a special operation of washing the preparation with water, a solvent or the like, and also, the preparation did not remain after the peeling (Comparative Examples 6 and 7).

As a generally known base for film-forming preparations, hydroxypropyl cellulose was selected instead of ethyl cellulose. A preparation was produced by the same method of that of Example 1, with the exception that efinaconazole, hydroxypropyl cellulose, ethanol, and a surfactant were mixed with one another at the ratio shown in Table 5.

TABLE 5

| | | Comp. Ex. 6 | Comp. Ex. 7 |
|---|---|---|---|
| Drug substance | Efinaconazole | 10 | 10 |
| Base | Hydroxypropyl cellulose | 16 | 16 |
| Volatile solvent | Ethanol | 74 | 64 |
| Surfactant | AMPHITOL 20AB | | 10 |

| | | |
|---|---|---|
| Film formation time | X | X |
| Peeling ability | X | ◯ |
| Surface stickiness | X | X |

When hydroxypropyl cellulose was used as a base, stickiness was observed on the surface of the preparation.

### Example 20

The results obtained by measuring the amount of the drug permeating through nails are shown in Table 6.

TABLE 6

| | |
|---|---|
| Clenafin | 0.4 μg/cm² |
| Example 1 | 0.8 μg/cm² |

The amount of efinaconazole permeating through human nails in the preparation of Example 1 was approximately 2 times larger than the amount of Clenafin which is an existing preparation.

### Example 21

A preparation was produced by the same method as that of Example 1, with the exception that efinaconazole, ethyl cellulose, ethanol, triacetin, and a surfactant were mixed with one another at the ratio shown in Table 7.

### Examples 22 and 23

A preparation was produced by the same method as that of Example 1, with the exception that efinaconazole, ethyl cellulose, ethanol, and a surfactant were mixed with one another at the ratio shown in Table 7.

TABLE 7

| | | Example 21 | Example 22 | Example 23 |
|---|---|---|---|---|
| Drug | Efinaconazale | 10 | 10 | 10 |
| substance | | | | |
| Base | Ethyl | 16 | 16 | 16 |
| | cellulose A | | | |
| Volatile | ethanol | 61 | 73 | 71 |
| solvent | | | | |
| Surfactant | Polyoxyethylene | 3 | 1 | 3 |
| | lauryl ether | | | |
| | sodium phosphate | | | |
| Ester | Triacetin | 10 | | |

| | | | |
|---|---|---|---|
| Film formation time | ◯ | ◯ | ◯ |
| Peeling ability | ◯ | ◯ | ◯ |
| Surface stickiness | Δ | ◯ | ◯ |

In Example 21, in which an ester was comprised in the preparation in amount of 10% by mass with respect to the total amount of the components in the preparation, slight stickiness was observed on the surface, but such stickiness was at a level causing no practical problems.

In Examples 22 and 23, in which a surfactant was comprised in the preparation in amount of 1% by mass or 3% by mass with respect to the total amount of the components in the preparation, both of the preparations were found to have no stickiness on the surface, and the preparations could be removed without performing a special operation of washing the preparation with water, a solvent or the like, and also, the preparations did not remain after the peeling.

### Example 24

The results obtained by measuring the amount of the drug permeating through nails are shown in Table 8.

TABLE 8

| | |
|---|---|
| Clenafin | 0.135 μg/cm² |
| Example 9 | 0.535 μg/cm² |

1. A therapeutic preparation for onychomycosis, comprising:
a) efinaconazole or a salt thereof,
b) 1% by weight or more and 30% by weight or less of ethyl cellulose,
c) 20% by weight or more of a volatile solvent, wherein the volatile solvent is water or a liquid component having a boiling point lower than a boiling point of water, and
d) 0.01% by weight or more and less than 30% by weight of a surfactant.

2-3. (canceled)

4. The preparation according to claim 1, wherein an amount of the ethyl cellulose in the preparation is 5% by weight or more and 25% by weight or less.

5. (canceled)

6. The preparation according to claim 1, wherein an amount of the efinaconazole or a salt thereof in the preparation is 0.1% by weight or more and 30% by weight or less.

7-8. (canceled)

9. The preparation according to claim 1, wherein the surfactant comprises a cationic surfactant.

10. The preparation according to claim 9, wherein the cationic surfactant comprises N-coconut oil fatty acid acyl-L-arginine-ethyl DL-pyrrolidone carboxylate.

11. The preparation according to claim 1, wherein the surfactant comprises an anionic surfactant.

12. The preparation according to claim 1, wherein the surfactant comprises an amphoteric surfactant.

13. The preparation according to claim 1, wherein the surfactant comprises a nonionic surfactant.

14. The preparation according to claim 1, wherein the surfactant has a glycerin backbone or a propylene glycol backbone.

15. The preparation according to claim 1, wherein the surfactant has a sorbitan backbone.

16. The preparation according to claim 1, wherein the volatile solvent comprises at least one selected from the group consisting of ethanol, isopropanol, ethyl acetate, acetone, diethyl ether, dimethyl ether, and water.

17. The preparation according to claim 1, wherein the volatile solvent comprises at least one volatile solvent other than water.

18. The preparation according to claim 17, wherein an amount of the at least one volatile solvent other than water in the preparation is 50% by weight or more and 90% by weight or less.

19. The preparation according to claim 1, further comprising a non-volatile component other than the surfactant.

20. The preparation according to claim 19, wherein an amount of the non-volatile component other than the surfactant in the preparation is 7% by weight or less.

21. The preparation according to claim 19, wherein the non-volatile component other than the surfactant comprises an ester.

22. The preparation according to claim 19, wherein the non-volatile component other than the surfactant comprises an alcohol.

23. The preparation according to claim 1, which is in the form of a liquid, and is capable of forming a film when solidified.

24. A preparation., comprising:
a) a pharmaceutically active ingredient,
b) ethyl cellulose,
c) 20% by weight or more of a volatile solvent, wherein the volatile solvent is water or a liquid component having a boiling point lower than a boiling point of water, and
d) a cationic surfactant.

25. The preparation according to claim 24, wherein the cationic surfactant comprises N-coconut oil fatty acid acyl-L-arginine-ethyl DL-pyrrolidone carboxylate.
